(19) United States
(12) Patent Application Publication
(10) Pub. No.: US 2026/0185989 A1
(43) Pub. Date: Jul. 2, 2026

(54) PROCEDURES FOR SEPARATING BIOLOGICAL SAMPLES USING DENSE IMMUNOMAGNETIC BEADS

(71) Applicant: Raven Biomaterials, Bethlehem, PA (US)

(72) Inventors: Caroline Hoedemaker, Bethlehem, PA (US); Thomas Russell, Doylestown, PA (US); Gary D. Fletcher, Media, PA (US)

(73) Assignee: Raven Biomaterials, Bethlehem, PA (US)

(21) Appl. No.: 19/129,113

(22) PCT Filed: Nov. 10, 2023
(86) PCT No.: PCT/US2023/037159

§ 371 Date: May 12, 2025

Related U.S. Application Data
(60) Provisional application No. 63/424,369, filed on Nov. 10, 2022.

(51) Int. Cl. G01N 33/543; C12N 5/00; G01N 33/50; G01N 33/53

(52) U.S. Cl. CPC G01N 33/54326; C12N 5/0087; G01N 33/5005; G01N 33/5306; G01N 33/54393

(57) ABSTRACT

Analysis of cell populations for both research and clinical applications such as cell therapy is often performed on whole blood that is close to 24 hours old. The reason for this is that the analysis site is removed from the blood draw site so that blood must be shipped overnight to the analysis site. During the 24-hour shipping period, granulocytes and other blood components break down into undesirable cell debris including nucleic acids that are released into the blood. Such debris is known to interfere with immunological assays and with thawing following freezing of samples. The present invention solves this issue by removing granulocytes (and optionally other components) using an anti-CD15 molecule (or other appropriate molecule) bound to metallic magnetic particles. Following mixing, the granulocytes (or other components) are removed and prepared for shipment to the analysis site or for other reasons.

The present invention claims priority under 35 USC 119(e) and under 35 USC 371 to U.S. Provisional Application No. 63/424,369 filed Nov. 10, 2022, the entire contents of which is incorporated by reference in its entirety.

### FIELD OF THE INVENTION

This invention relates broadly to the specific and different challenges regarding the separation of biological sample components using solid phase dense, metallic magnetic particles and their use to very rapidly remove cells or biological material such as granulocytes and/or platelets, both of which are known to adversely affect sample quality. The invention is enabled by the availability of a magnetic bead with specifically different properties from the beads that are currently available enabling separation in more complex solutions, and over greater distances. The present invention contemplates equipment and properties that enable use with a “denser” immunomagnetic bead, and contemplates equipment capability enabled by the use of a bead that has a higher magnetic susceptibility than is common in the market place today. The present invention is embodied by both means and apparati that permit removal of certain undesired cells at a blood draw site from undiluted blood prior to shipment, or for general separation purposes.

### BACKGROUND OF THE INVENTION

### Description of Related Art

Immunological monitoring is a critical component in both basic research and clinical research especially for studies related to cell therapy/transplantation. The majority of samples for immunological monitoring are derived from peripheral blood mononuclear cells (PBMC) within whole blood (WB), apheresis based materials typically in a leukopak (LP), or morcellated samples of solid tissue (MST). For purposes here we use WB as the lead example, and refer to any of these as “blood products”.

In one example where this is applicable, the WB samples are drawn into a blood bag and the blood is processed in a laboratory to remove most of the red blood cells and granulocytes via a Ficoll gradient separation. Often these samples are then frozen, or transported overnight to a laboratory. This process is labor intensive, requiring dedicated laboratory space, equipment, and trained lab personnel. In another example, the majority of blood samples that are to undergo cell manipulation are obtained via the Leukophoresis process (which is a procedure that generally separates and collects white blood cells and is often the first step in chimeric antigen receptor treatments).

More often, the only solution for processing the blood sample is to ship blood products to a processing center for PBMC or other cell isolation and freezing. The use of the cells for their immunogenic value, or their immune monitoring occurs at a later point in time. Freeze/thaw processes often lead to significant cell losses and decreased viability. The functional integrity of the blood cells declines with time from blood draw, and it is well established that the presence of granulocytes in the blood significantly contributes to this functional decline. The longer the storage time until processing blood, and particularly, whole blood, the higher the degradation/contamination degree of the sample with granulocytes. These long storage times adversely affect immunological functional studies i.e., using ELISPOT and intracellular cytokine analysis by Flow Cytometry. During prolonged storage, granulocytes become activated and change their buoyancy profile, which leads to their less efficient separation during isolation using the Ficoll process.

Activated granulocytes are known to suppress T-cell function by down-modulating the signal transducing zeta chain of the CD3 molecule. Therefore, depletion of granulocytes and/or procedures that will lead to effective granulocyte removal and inhibition of their activation and its inhibitory effects on T-cells shortly after blood draw would extend the functional integrity of PBMC samples and provide meaningful clinical immunological monitoring data. Thus, it is desired that an easy to integrate solution at the blood draw site be developed that has both controllable and minimal work-load.

Although magnetic bead technology manufactured by the likes of Miltenyi Biotec and ThermoFisher/DYNAL, has been known for some time and their technology may be able to remove granulocytes, the procedures they have outlined will not solve the problem at hand. The procedures developed by these manufacturers do not lend themselves to the rapid and direct removal of granulocytes in un-diluted whole blood. Accordingly, their procedures do not allow for the necessary removal of granulocytes or other cells at blood collection sites.

The procedures outlined by these manufacturers are not well adapted to the removal of granulocytes from un-diluted whole blood. The ThermoFisher website states the following: “Dynabeads can be added directly to undiluted blood if reduced cell isolation efficiency is tolerated . . . .” This is not desirable. Also, “When incubating Dynabeads and cells, the incubation temperature must be 2-8° C. to reduce phagocytic activity and other metabolic processes. The magnetic beads need to be incubated for 30 min (depletion) at 2-8° C. with gentle tilting and rotation”.

The Miltenyi Biotec website states the following: “CD15 MicroBeads were developed for depletion of CD15 positive cells from human lysed peripheral blood”. For shipping whole blood, lysing of the red cells is not an option and cannot be done conveniently at the blood draw site.

Also, the Miltenyi process requires columns for depletion of cells which results in significant loss of desired cells (e.g., PBMCs).

Thus, it should be apparent from the various manufacturers of magnetic beads that the magnetic particles of the art based on non-metallic iron oxide magnetic material simply are not suited or suitable for use at a blood draw site. They cannot be mixed into the cellular solution efficiently, as they exhibit a colloidal nature.

Moreover, in one embodiment, the beads of the prior art are less dense than the magnetic beads used in the method(s) described herein. Less dense beads suffer from forces impacting particle dispersion into biological mixtures including gravity, buoyancy, viscous drag, thermal, and electrostatic interactions, and are compounded by other material's (cells, proteins, etc.) properties such as surface tension and colloidal properties involving particle size and shape that prevents them from being properly mixed to remove the unwanted components in blood. The smaller, less dense beads of the prior art require dilution of the blood, using smaller sample sizes of blood, and/or vigorous physical mixing means (such as vortexing) to properly disperse the beads throughout the blood. They also require longer mixing/dispersion times to properly disperse the beads throughout the diluted blood thereby leading to longer processing times to remove the unwanted components.

Beads that are too dense may settle directly to the bottom, leaving insufficient time for binding of the unwanted components. These high density beads may also require mixing and/or vigorous physical agitation to properly disperse the beads throughout undiluted blood, and the mixing may still not allow for proper dispersion of the beads, thereby resulting in inefficient binding and inability to remove the undesired components (such as granulocytes).

### SUMMARY OF THE INVENTION

The invention, in its broadest sense, uses the magnetic beads of the present invention as disclosed herein as a means of separating components in a fluid. In an embodiment, the magnetic beads comprise a density, a mass, and a compositional make-up that makes them ideally suited for this purpose. In an embodiment, the present invention also relates to equipment and methodologies used to achieve this purpose.

In an embodiment, the present invention relates to containers in which the fluid can be held wherein the container is made of a material that allows a magnetic field to traverse the material. In an embodiment, the magnetic beads will be present in the interior of the container with the fluid, and the magnetic beads can be used to effectively separate one or more components in the container. In an embodiment, the magnetic beads will have a first component affixed to the magnetic beads wherein the first component binds a second component present in the fluid. In an embodiment, the magnetic beads can be uniformly dispersed in the fluid allowing the magnetic beads with the first component to effectively bind the second component. The magnetic beads can then be consolidated to a particular location in the container by applying a magnetic field to the beads, thereby allowing the separation of the beads from or within the container.

In an embodiment, the magnetic beads and their used by the present invention are of a density that allows the magnetic beads to be rapidly, uniformly dispersed in fluids/liquids that have viscosities in a range from 0.01 cP to 10 cP, and they are ideally suited for fluids/liquids that have a viscosity of greater than about 1 cP and less than about 6 cP.

In an embodiment, the invention disclosed herein provides methods and means (apparatus) to sterilely remove, for example, granulocytes or granulocytes plus platelets at a blood draw site or in the laboratory in volume. Other cellular permutations that can be used include retaining CD8 cells, but removing CD15, CD16, CD19, CD4 cells for example. Thus, the present invention relates to a plurality of potential uses.

One embodiment of the invention includes the use of dense, metallic, magnetic beads coupled to anti-granulocyte surface molecules (CD15) or anti-granulocyte surface molecules (CD15) and anti-platelet surface molecules (CD41 and/or CD61) that effectively bind to granulocytes or granulocytes plus platelets very rapidly in undiluted whole blood. In an embodiment, the granulocytes are then removed from the solution by magnetic separation. This can be accomplished in a container (e.g., blood bag) as disclosed herein. Blood from a patient is placed in a container (e.g., a blood bag), which is optionally mixed to “turbulently” move the dense beads throughout the bag using a single or a specific combination of mixing modalities, with the appropriate speed variations, for an appropriate time. In one variation, the motion is in a direction that opposes gravity. The blood bag, which comprises the undiluted blood and the magnetic beads with the appropriately attached molecule to bind cells such as granulocytes is then placed in a magnetic separation device that separates by magnetism the bead bound granulocytes from the undiluted blood. The blood bag in the magnetic field can be partially sealed to allow separation and isolation of the magnetic beads that comprise the molecule that binds granulocytes as well as the bound granulocytes. The partial sealing of the blood bag can allow any blood that is free of granulocytes that is adjacent to the magnetic beads to amass with the other granulocyte free blood in the bag, thereby reducing the loss of the granulocyte free blood. After separation, the magnetic beads can optionally be excised from the blood bag and the blood bag can be fully sealed for shipment to an external site.

In an embodiment, the mixing may occur via end over end mixing to prevent settling. The mixing may occur at fixed or variable speeds. In a variation, the mixing may occur via lateral mixing where the mixing is spread throughout the blood product and this mixing too, may occur at a fixed or a variable speed. In a variation, mixing may occur via the use of an alternate substance in the blood bag. For example, if the blood bag containing the undiluted blood has been filled using a vacuum process, a gas may be added to create a head space to allow a sparging type of mixing.

The volume of undiluted blood in the blood bag may be less than 400 ml so as to allow a volume of gas to be introduced into the headspace in a blood bag with a 500 ml capacity so that, as the gas that is introduced into the headspace, the gas does not contact the blood. Alternatively, the headspace may be decreased so as to be able to introduce the gas into the blood directly. If gas is to be used for sparging, a thicker heat sealable plastic blood bag may be used to accommodate increased pressures (from the sparging gas). The gas may be an inert gas or alternatively, it may be air or some other non-inert gas.

In another embodiment, the particle bound granulocytes are removed by gravity separation due to the density of the particles. In a variation, both magnetic and gravity separation occur. In an embodiment, centrifugation may be used to aid in separation of the granulocytes (wherein the dense magnetic beads will settle to the bottom of the blood bag). In an embodiment, the granulocytes can be removed directly in a blood bag.

In an embodiment, the magnetic beads as disclosed herein provide the ideal vehicle for separation of unwanted (or wanted) components from undiluted blood because the density and the magnetic field strength of the beads are greater than those of the prior art. The higher density (but not too high) leads to better and more equal dispersion of the beads throughout the fluid, and the higher magnetic field strength of the beads allows for rapid and non-degradative separation and isolation methodologies to be used, as well as larger quantities of fluid to be processed. Larger quantities can be processed because the higher magnetic field strength of the beads allow a magnetic field to penetrate further into the fluid, thereby providing better separation than would be attained by beads with a lower field strength. Magnetic properties decrease by the square of the distance.

In an embodiment, the magnetic field strength, applied directly form a magnet to the beads, may be sufficient so as to allow separation over distances greater than 6 mm and up to 3 cm or 4 cm or 5 cm4 cm in distance.

### BRIEF DESCRIPTIONS OF DRAWINGS

FIG. 1. High cell recovery of non-targeted cell populations. The top figure is the light scatter histogram of PBMCs run on a Becton Dickinson Flow Cytometer. Y-axis: side light scatter (90 degree); X-axis: forward light scatter. Three cell populations are easily distinguished: Top: granulocytes; Middle: monocytes; Bottom: lymphocytes. The bottom figure represents the light scatter histogram of PBMCs following the depletion of granulocytes using CD15 RBI-beads using the method described herein.

FIG. 2. Rapid removal of granulocytes using anti-CD15 metallic, magnetic particles. Whole blood was analyzed on a Beckman-Coulter 3-part differential analyzer. Three cell populations can be distinguished: Top left figure (Control): Left: lymphocytes; Middle: monocytes; Right: granulocytes. The top right, bottom left and bottom right figures demonstrate the removal of CD15 positive granulocytes using the method described herein as a function of incubation time with the CD15 metallic, magnetic particles. Incubation time ranged from 1 minute to 3 seconds.

FIG. 3. Effect of CD15 depletion on various cell populations present in whole blood. Anti-CD15 RBI-beads of the invention were used to deplete granulocytes and the effect of the depletion on lymphocyte subset populations (CD3, CD4 and CD8) was examined.

FIG. 4 depicts a variable speed end over end mixture, on top of a lateral mixer, with a blood bag shown in the mixer.

FIG. 5 depicts a close-up of the rotating portion of the end over end mixer with the blood bag, and separation plate using an array of magnets.

FIG. 6 depicts an image showing the beads with a common lab magnet at the top showing the ability of the magnet to attract the beads at a distance of between 3.5 cm (2 cm for the microcentrifuge tube holder+1.5-2 cm measurement).

FIG. 7 depicts another image of a end over end mixer with a mother board that controls the variable speeds.

FIG. 8 depicts an image showing the pattern of the magnetic beads in a container (e.g., a blood bag) when the mixing conditions have created a dispersion of the beads FIG. 9 depicts an image showing the pattern of the magnetic beads in a container (e.g., a blood bag) when the mixing conditions are constant.

FIG. 10 depicts another image showing the pattern of the magnetic beads in a container (e.g., a blood bag) when the mixing conditions are constant, at a different condition.

### DETAILED DESCRIPTION OF THE INVENTION

The present invention relates to the use of magnetic beads as disclosed herein as a means of separating components in a fluid. In an embodiment, the magnetic beads comprise a density, a mass, and a compositional make-up that makes them ideally suited for this purpose. In an embodiment, the present invention also relates to equipment and methodologies used to achieve this purpose.

In an embodiment, the magnetic beads of the present invention have a density that allows the magnetic beads to be uniformly dispersed in fluids (or liquids). The magnetic beads of the present invention are superior to the beads of the prior art because the beads can be uniformly dispersed in fluids that have greater viscosity than the beads of the prior art. For example, blood, which has a viscosity of 3.5-5.5 cPoise (cP) does not allow the beads of the prior art to be uniformly dispersed throughout blood because the density of the prior art beads are not sufficiently different from blood based materials to allow their movement through the relatively viscous blood. Accordingly, the beads of the prior art have poor separation capabilities in fluids that have moderate to moderately high to high viscosities.

The magnetic beads of the invention can also be used with plasma or other fluids as described herein. Plasma ordinarily has a viscosity of approximately 1.10-1.30 mPa·s (i.e., 1.10 to 1.30 cP). Other fluids that can be used in connection with the beads of the present invention include whole blood, diluted blood, PBMCs (peripheral blood mononuclear cells) of various concentrations, leukoblasts, tumor homogenates (e.g., solid tumors that have been blended), liquid-based cytology samples and other fluids. It should be understood that the magnetic beads of the present invention can be used for any of a plurality of purposes such as to isolate natural products from a compositional mixture.

Moreover, the density and the compositional makeup of the magnetic beads of the present invention (i.e., a solid metal core with a thin film attached to an agent that binds to a biological substance) allow the magnetic beads to not only be uniformly dispersed in fluids, but they also allow the magnetic beads to be readily moved at distances that are between 5 mm and 4 cm from the source magnetic field applied to the magnetic bead. The combination of these properties make the magnetic beads and equipment needed to use these beads in the present invention uniquely suited to separating components in fluids, and unequivocally superior to the beads of the prior art.

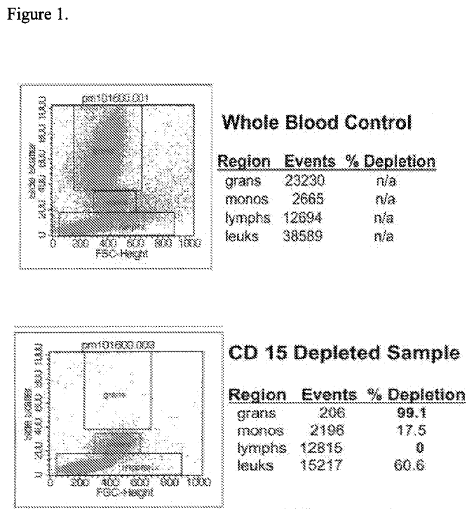

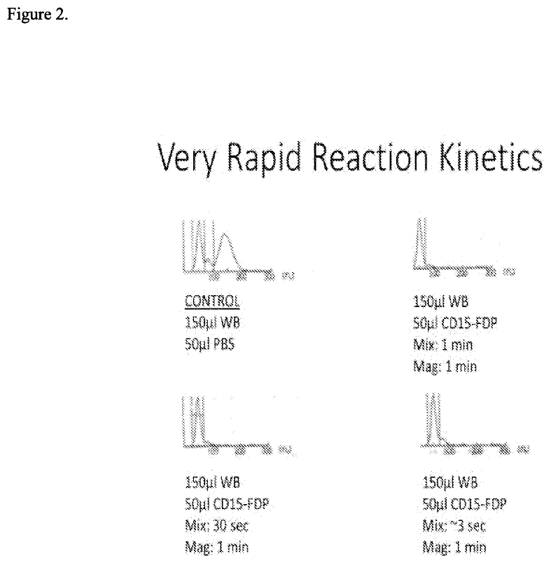

In an embodiment, the magnetic beads can be used with blood to remove granulocytes. In order to provide granulocyte free blood for overnight shipment a procedure is needed that can be performed at the blood draw site with minimal involvement by laboratory personnel. In an embodiment, dense, metallic, magnetic particles can be used that allow one to meet this requirement, and these dense, metallic, magnetic particles are described in U.S. Pat. No. 9,435,799, which is herein incorporated by reference in its entirety. The metallic magnetic particle is as described in U.S. Pat. No. 9,435,799. These particles meet all requirements for working at a blood draw site: they work directly in undiluted whole blood, do not bind or trap cells of interest yielding close to 100% of recovery of non-targeted cells, in this case PBMC (FIG. 1), and the particles bind to the targeted cells rapidly on the order of seconds to minutes (FIG. 2). Thus, the separation of the non-desired cells can be rapidly removed from undiluted blood.

The optimum bead size, reaction time and magnetic separation time is determined as is described in U.S. Pat. No. 9,435,799.

The best bead size will be determined using particles in the size range from about 0.5 micron to 3.5 micron but not limited thereto. For magnetic separation, the beads used will be as described in U.S. Pat. No. 9,435,799. In a variation, any magnetic metal bead may be used in the processes/methods disclosed herein. The bead can be manufactured as disclosed in U.S. Pat. No. 9,435,799 or, in a variation, obtained commercially from sources such as Sigma or Novamet.

The density of the particles can be in the range of 2-20 g/cc or 4-10 g/cc. In a variation, the density of the particles can be in the range of 4-10 g/cc or 4-9 g/cc or 6-9 g/cc.

For removal of granulocytes from undiluted whole blood, anti-CD15 antibodies can be coupled to the magnetic bead by means known in the art including direct adsorption, ionic coupling, or covalent coupling. Though anti-CD15 monoclonal antibody is preferred, it should be understood that any monoclonal or polyclonal antibody bound to the metallic magnetic bead that removes granulocytes is contemplated and considered to be included in the disclosure. In an embodiment, and as disclosed above, the metallic beads may also include beads that remove platelets which are known to be sticky and therefore may also need to be removed at the blood draw site to provide the best material for overnight shipping. In an embodiment, the present invention relates to using the beads as disclosed herein in a blood bag and quantitatively testing the undiluted blood to ascertain if the removal of granulocytes is sufficient for preparing blood for shipment. In an alternate embodiment, the present invention relates to the removal of granulocytes and/or platelets to prepare the whole blood sample for shipment.

In one embodiment, the manual method for removal of granulocytes is summarized here:

The blood bag disclosed herein will have magnetic beads contained therein, which obviates the need for the user to add beads or blood manually to the undiluted blood, thereby making the procedure convenient for use at blood draw sites prior to shipment. For the purpose of clarification, the anti-CD15 and/or anti-platelet magnetic particles i.e., CD41 and/or CD61 will be in a blood bag. The beads in the blood bag can be in a liquid state or a lyophilized state. Once the undiluted blood has been transferred to the blood bag the manual method can proceed as described:

### Method

1. Identify and use a patient whose whole blood (WB) needs to be analyzed/collected.

2. Insert WB into the blood bag containing the magnetic beads with the appropriately attached cell binding molecule that is designed to bind granulocytes (or any other desired molecule/cell to be isolated);

3. Mix the blood bag on such as an end-over-end mixer* or lateral mixer at customized setting for up to 5 minutes (actual time may vary and the best time will be determined experimentally);

4. Place the blood bag in magnetic field for 1 minute to allow the magnetic beads with the attached granulocytes to migrate to an end or corner of the blood bag (the actual magnetic separation time may vary and can be determined experimentally);

5. Partially seal the blood bag while the blood bag is still in the magnetic field, allowing blood components that are free of granulocytes that are in the corners/ends of the blood bag to migrate to and amass with the other blood in the blood bag that is substantially free of the granulocyte and/or granulocyte plus platelet depleted blood;

6. Optionally excise (and recycle) the magnetic beads containing the bound granulocytes (or platelets); and

7. Fully seal the blood bag and prepare the blood bag for shipment.

### Equipment Required:

In an embodiment, the following equipment may be used.

*Mixer: Due to the approximately 5-fold difference in density between CD15 particles and cells, proper mixing should occur to ensure contact between the particles and the targeted cells to be removed/isolated.

In an embodiment, mixing may be accomplished by end-over-end mixing using e.g., an ATR Rotomix mixer with variable speed. In an embodiment, the mixing speed may be 8-50 rpm or 10-30 rpm.

In an embodiment, it has been found that in order to get good uniform dispersion of the beads in a fluid, that variable mixing provides the best avenue for attaining the uniform mixing. Mixing that tends to be very consistent and does not have variability to it, leads to bead dispersion that is less uniform with beads tending to congregate in certain areas of the fluid. Thus, an end over end mixer that has a variable speed setting or a magnetic stirrer that has a setting that allows for non-uniform speeds provide the ideal mixers that allow for uniform dispersion of the magnetic beads throughout the entire fluid matrix. Accordingly, the use of a mixer with variable speeds also leads to better separation of components in a mix (in addition to higher yields of that component), better purification protocols and other advantages.

### Magnetic Separation:

a. In an embodiment, the magnets for use with anti-CD15 magnetic particles disclosed herein can be obtained from any of a variety of sources. In an embodiment, magnets are available for sample volumes from approximately 0.5 mL to 500 mL or with sample volumes between 450 ml and 500 ml. It should be understood that if larger volumes are to be used, the magnet should be chosen so as to allow the correct separation/isolation of the magnetic beads with the bound granulocytes.

b. Magnets from suppliers of superparamagnetic particles will also work as long as while placed in the magnetic field the blood bag experiences a sufficient magnetic field to allow isolation/separation of the beads

c. Manufactures of magnetic separation devices such as Life Sep, but not limited thereto, can by means known in the art manufacture a magnetic separation device that is compatible with the blood bags as described and disclosed herein.

In another embodiment, dense particles that settle by gravity as disclosed in U.S. Pat. Nos. 5,576,185 and 9,435,799 can be used in the blood bags as disclosed herein. The particles are dense (4-10 g/cc) and can be composed of magnetic or non-magnetic material. An advantage of magnetic particles is simply that following gravity settling a magnet can be placed at the bottom (corner, or side) of the blood bag to hold the particles in place prior to sealing the bag.

The following non-limiting examples will demonstrate the key attributes of the technology that will enable the blood bag disclosed herein to provide samples depleted of undesired cells such as granulocytes and platelets at a blood draw site prior to shipment of the sample to external sites for analysis. The method and apparatus disclosed herein should be convenient and easy to use by trained laboratory personnel at the blood draw site. The following examples demonstrate that this is the case for the invention disclosed herein.

### EXAMPLES

### 1. High Cell Recovery of Non-Targeted Cell Populations

In an embodiment, it is desired that the technology used to substantially deplete undesired cells prior to shipment to external sites. In a variation, the technology works in undiluted whole blood at room temperature (cooling is not required).

Whole blood was incubated with the CD15 magnetic particles disclosed herein by the method disclosed herein to remove granulocytes. The depleted and control non-depleted samples were analyzed on a Becton Dickinson Flow Cytometer (Control; FIG. 1; Top). The analysis parameters were forward and 90-degree light scatter. The histogram shows 3 populations of cells: granulocytes (top), monocytes (middle) and lymphocytes (bottom). Following CD15 depletion (FIG. 1; bottom), the results demonstrate the effective removal of granulocytes with good recovery of the desired cells, in this case lymphocytes. The slight depletion of monocytes in the sample was attributed to a subset of monocytes that express the CD15 antigen. These results demonstrate the desired results using the magnetic particles disclosed herein: excellent depletion of a non-desired cell population (granulocytes) with good recovery of a desired cell population (lymphocytes).

### 2. Rapid Removal of Granulocytes Using Anti-CD15 Metallic, Magnetic Particles

In an embodiment, the present invention relates to the binding of CD15 magnetic particles to granulocytes and the removal of the particle bound granulocytes by a magnetic field in short periods of time. The results disclosed in FIG. 2 show the relatively rapid removal of granulocytes by the magnetic particles. Whole blood was incubated with CD15 magnetic particles disclosed herein by the method disclosed herein to remove granulocytes. The samples were analyzed on a Coulter 3-part differential hematology analyzer which yields lymphocytes (left peak), monocytes (middle peak) and granulocytes (right peak) as demonstrated in the control sample (FIG. 2; top left histogram). FIG. 2: top right, bottom left and bottom right demonstrate very rapid binding times with a magnetic separation time of 1 minute. These results demonstrate very rapid mixing times for granulocytes with the CD15 antigen on their cell surface. For isolating granulocytes (or platelets) from undiluted blood in a blood bag (with larger volumes that may approach 500 ml), the number of magnetic beads with the molecule designed to bind the granulocytes may be increased to allow a higher concentration of the dispersed beads in the sample (thereby having more beads to bind the granulocytes). In a variation, the mixing time may be increased to allow sufficient binding of the granulocytes to the beads. In a variation, the mixing may be more vigorous so as to aid in dispersion of in the sample. In an embodiment, the beads may be added as aliquots so that the beads with the appropriate granulocyte binding molecule bind the granulocytes, and then subsequently another aliquot of the beads can be added (in a “second round”) to bind more granulocytes. By using this sequential method of addition of the beads (with optional magnetic separation/isolation steps after each mixing occurs), one can remove many of the granulocytes in a first round and remove more in a second round. In an embodiment, the magnetic separation time may also be adjusted. The results shown in FIG. 2 demonstrate that very rapid mixing and magnetic separation times gives good separation so it is contemplated that this can be used by laboratory personnel at the blood draw site.

### 3. Effect of CD15 Depletion on Various Cell Populations Present in Whole Blood

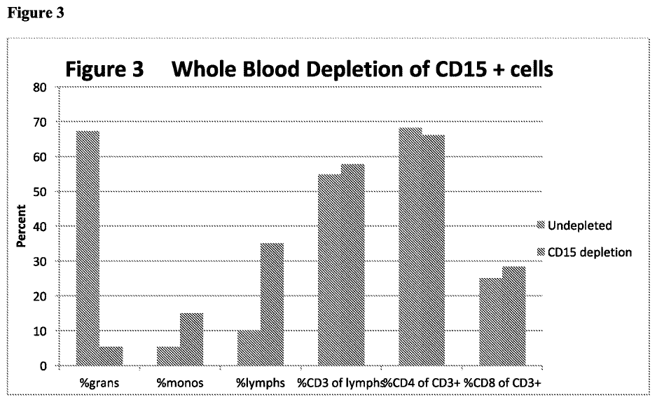

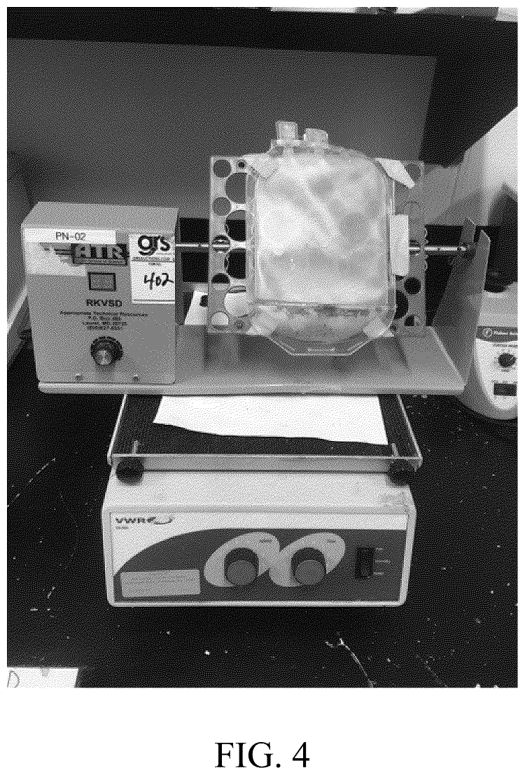

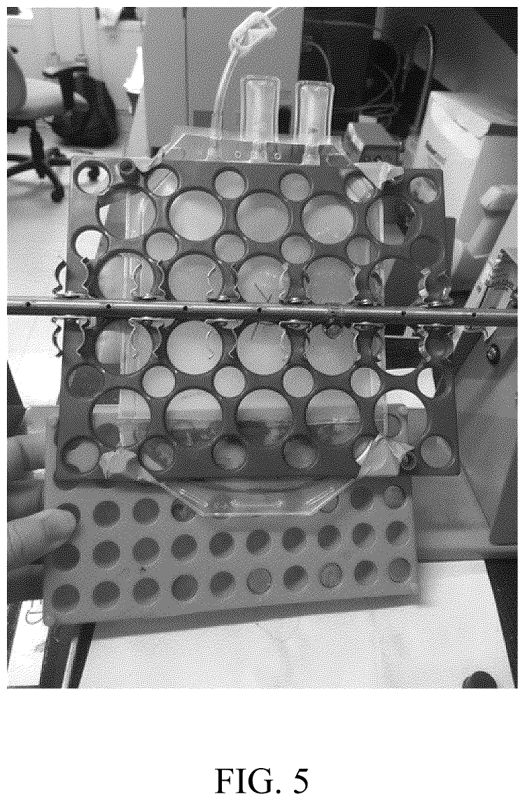

In an embodiment, the granulocytes are removed prior to shipment of blood to external sites and, following the depletion of CD15 positive cells using the magnetic particle separation technology described herein, the remaining PBMCs are not altered. Referring to FIG. 3, the removal of granulocytes did not adversely affect the following lymphocyte populations: CD3, CD3/CD4 and CD3/CD8 populations. CD15 positive cells were removed by the method described herein and the whole blood was analyzed by dual color Flow Cytometry.

The depletion of specific cell types based on surface expressing antigens will also deplete extracellular vesicles (EV) expressing those antigens. For example, platelet EV will also be cleared when CD41 beads are used to deplete platelets. This provides an ideal avenue to be a significant advantage in the field of “liquid biopsy” wherein circulating tumor EV can be being detected as the platelet EV makeup a significant fraction of the total EV population. In fact, EV analysis is a rapidly developing field. Debulking EV is as important as debulking the cellular populations. In an embodiment, the purification of exosomes, a subset of the EV, are accomplished by positive selection based on CD9, CD63, and CD81 from platelet free plasma. Platelets and platelet derived EVs express CD9 and therefore, even though the plasma is platelet free, a significant fraction of the exosomes will be derived from platelets.

### 4. The Objective is to Determine the Influence of Different Mixing Methods Available on Settling Time of Cores (the Magnetic Beads).

The materials used were as follows:
Core material (A,B,C,D),
2 mL test tubes,
1% BSA/PBS buffer,
Mixing equipment (EOE mixer, vortex, lateral mixer)

The procedure was:

1. Add ˜20 mg of core material to 2.0 ml test tube.

2. Add 1.5 ml of 1% BSA/PBS.

3. Disperse the core throughout the material by one of four methods
a. Simply flicking the tube,
b. End over end mixing, for 5 minutes,
c. Vortexing, for 30 s.d. Lateral mixing, for 3 minutes

Data/Observations are presented in table 1A.

TABLE 1A

| | Settling Times for Each Core | | | |
|---|---|---|---|---|
| Condition | A | B | C | D |
| a. No stirring (simple flick)* | 11:52 | 17:52 | 2:09 | 3:03 |
| b. End over end mixing, 5 minutes | 10:41 | 16:22 | 2:00 | 3:05 |
| c. Vortexing, 30 s | 10:51 | 14:12 | 3:21 | 3:12 |
| d. Lateral mixing, 3 min** | 8:12 | 13:20 | 1:29 | 2:33 |

It should be noted that simple flicking (no stirring) resulted in a large majority of particles settling to the bottom quickly. The measured time is time taken for solution to become clear.

For the lateral mixing, without being bound by theory, it was believed that the faster settling time is likely due to lateral mixing having no effect on upward dispersion, with settling occurring in the meantime.

It was thus concluded that the mixing method has little influence on the time it takes for core to settle in most cases. Core B seemed to settle faster after vortexing than other methods.

### 5. A Test was Done to Investigate how a Sample of Core Settles Under Lateral Mixing at Different Speeds in Bags.

The materials and reagents used were as shown in Table 1B.

TABLE 1B

| PN | Name | Lot # |
|---|---|---|
| 300001 | Core A | 25 |
| 300002 | Core B | 16 |
| 300003 | Core C | 17 |
| 300004 | Core D | 18 |
| IV058640 | SimRx | — |
| | Simulated | |
| | Blood bags | |
| | Disposable | |
| | Pipettes | |

For this test, the equipment used was
VWR Orbital Shaker,
ATR End over End mixer, and
Stopwatch.

The method was as follows:

Agitate a mixture of fluid (PBS/BSA) and RBI-core and note the settling pattern after lateral mixing.

1. Cut the outlet/inlet tube on a simulated blood bag short.

2. Pour 50-100 ml of TRIS buffer solution into a tube or beaker.

3. Add 50 mg of core material to the container.

4. Agitate the mixture using an end over end mixer or just by shaking it.

5. Pipette out solution immediately after shaking, trying to get as much core in the pipette as possible.

6. Place the mixture in pipette into a simulated blood bag. (Bag is 4¾″ by 7″ (interior dimensions)).

7. Repeat as necessary to get all the core material.

8. If any core material left was left in the container, add TRIS buffer and repeat the whole process.

9. Manually Shake/stir to obtain a visually even mixture.

Subsequently, the simulated blood bag was placed on a lateral mixer at a set speed and time was recorded and the bag was observed. Beads were tested at both a constant speed and a variable speed.
1. Constant Speed 2, 4, 6—Beads A, B, C, D
2. Variable Speed.

Table 1C summarizes the results:

TABLE 1C

| Core | Speed | Findings |
|---|---|---|
| A | Constant speed 2, 4, 6 | Spiral pattern at lower speeds (<=2). |
| | | Even dispersion at higher speeds (>=6). |
| | | At speeds >8, BSA is denatured, and bubbles are formed. |
| A | Variable speed | Particles settle quickly when speed drops. Switching between speeds of |
| | | 4-6 give a consistent dispersion/deposition pattern. Other speeds give |
| | | less dramatic results. |
| B | Constant speed 2, 4, 6 | Clumps form at lower speeds (<=2). |
| | | At medium to higher speeds, dispersion is achieved, although most |
| | | heavier particles stay close to the center. Fewer heavier particles at the |
| | | center at higher speeds. |
| | | This indicates B core is heavy. |
| B | Variable speed | Streaks of smaller particles settle when moving from high (>=6) to |
| | | medium or lower speeds. Most heavier core sticks stubbornly to the |
| | | center. This indicates B core particles are too heavy to be carried about |
| | | in solution, and that they would settle quickly given the chance. |
| C | Constant speed 2, 4, 6 | Odd patterns are formed at lower speeds. (I have no explanation for |
| | | them). |
| | | At medium speeds, particles settle quickly in the usual spiral shape. |
| | | Continual resuspension and deposition occur. At speeds 6-8, particles |
| | | are evenly distributed in solution. However, at speed 9-10, particles |
| | | stream to the center. (Still looking for explanation). |
| C | Variable speed | Consistent resuspension and deposition achieved using faster to |
| | | medium speeds. |
| | | Particles seem to recover their initial patterns when speeds change (e.g., |
| | | when speed changes from 4 to 2, the odd triangular pattern formed at |
| | | speed 2 normally holds again). This happens very quickly. This |
| | | indicates that particle movement is largely independent of changes in |
| | | speed. |
| D | Constant speed 2, 4, 6 | Note that in experiment D particles were far fewer, so they were harder |
| | | to observe patterns. |
| | | Slower speeds form stripes close to the centre. Faster speeds form |
| | | spirals further away from the centre. |
| D | Variable speed | Inconsistent patterns noticed. Larger spirals formed only on speed 2. |
| | | This might indicate that most of the material keeps swirling at all |
| | | speeds higher than 2. |

The conclusions that were drawn from this experiment was:
Faster speeds seem to be better for dispersing particles in the liquid.
Cycling through speeds of 4 to 6 should give a healthy balance and a uniform distribution of core at the center and out wide.
At much higher speeds (8-10), the BSA was denatured and released bubbles. Note for other organic materials in blood.

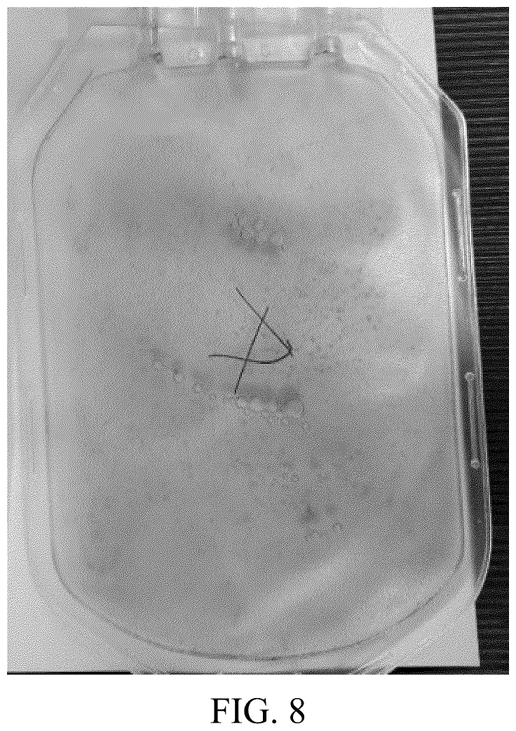

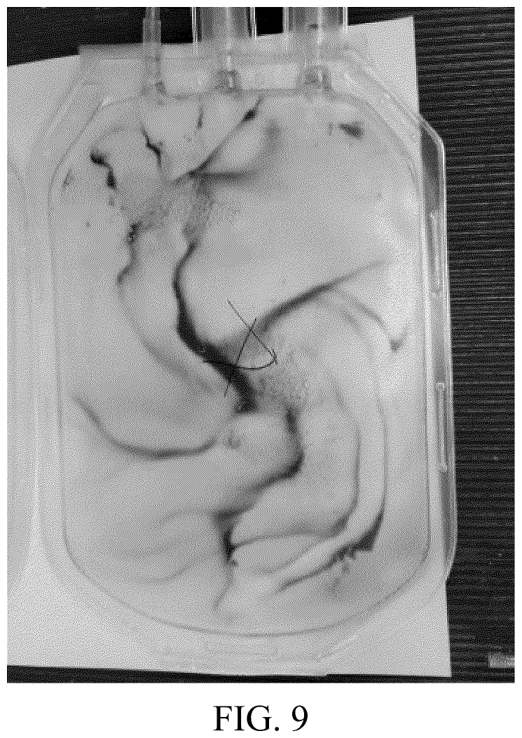

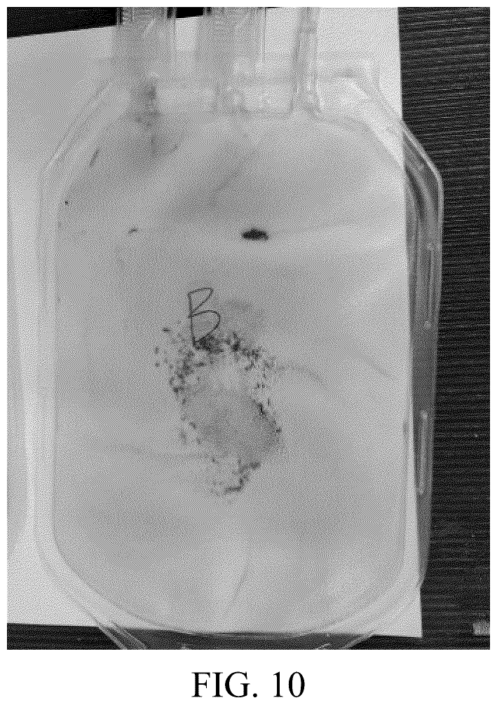

FIGS. 8-10 show representative patterns seen for some of the magnetic beads that were mixed in the above experiments. The magnetic beads in FIG. 8 are uniformly dispersed throughout the medium because the mixing was performed rather vigorously, but also mixed at variable speeds. The magnetic beads in FIG. 8 give very good isolation/purification/separation of components due to the uniform dispersion of the magnetic beads. The magnetic beads in FIG. 9 show some congregation of the beads due to constant (unvaried) mixing and a constant lateral motion meaning that separation and isolation of the targeted component(s) will be less ideal than the beads mixed at variable speeds. In FIG. 9, the beads remained in this pattern for 20 minutes. FIG. 10 shows an alternative bead patter with different sized particles and constant mixing, wherein the magnetic beads tend to clump in the center (i.e., they are not uniformly dispersed).

In an embodiment, the present invention relates to compositions and processes that allow for the ability to remove granulocytes from undiluted blood while the undiluted blood is in a blood bag. Being able to remove granulocytes (or any undesired component) while it is in a blood bag reduces transfers to other devices/containers/compartments. This allows for a reduction in the amount of material lost while undergoing one or more transfers, allows for a reduction in cost as fewer containers are necessary, and also allows for a reduction in the time it takes to remove the unwanted material (e.g., granulocytes).

It should be understood that although the compositions and processes are described with reference to granulocytes, the process can be used (and compositions can be appropriately designed) that will remove any undesired component/molecule in blood (or any fluid) using the magnetic beads and the methodologies described herein. By simply modifying the composition so that a first molecule that has an affinity for a second undesired molecule is adhered to the magnetic beads, the second undesired molecule can be removed from the fluid (e.g., blood, urine, or other biological fluid sample).

For efficient processing, beads must be easily dispersed throughout the sample to permit all of the cells adequate opportunity to bind to the antibody-coated bead. The forces impacting particle dispersion into biological mixtures include gravity, buoyancy, viscous drag, thermal, and electrostatic interactions and proper dispersion is compounded by other material's (cells, proteins, etc.) properties such as surface tension and colloidal properties involving particle size and shape.

The magnetic beads as described herein, in one embodiment have significantly higher densities than the beads of the prior art allowing them to properly mix. With the denser beads, gravity dominates the other forces alluded to above, thereby making mixing easier. The denser magnetic beads do not suffer from the drawbacks seen with the smaller superparamagnetic nanobeads, in which the non-gravitational forces dominate the gravitational forces, thereby hindering mixing. In order for the less dense beads to disperse throughout a fluid mixture (such as blood), dilution of the cellular mixture may be required, and incubation periods for the beads to disperse throughout the mixture must be increased.

Furthermore, if the particles are too dense, they settle too quickly to permit adequate time for cellular binding.

Table 1 shows a comparison of the beads that are ideally suited for blood dispersion, binding and separation relative to the less dense paramagnetic beads, as well as some of the other currently available technologies

TABLE 1
In an embodiment, RBI beads are the magnetic beads used herein.

| Product | | Properties | | | | | |
|---|---|---|---|---|---|---|---|
| Category | Company | Mixing | Magnetic | Speed | Surface | Purity | Recovery |
| RBI Beads | | Good | High | Fast | Simple | High | High |
| Super | Miltenyl, ThermoFisher | Poor | Low | Slow | Complex | High | Low |
| paramagnetic | BD, StemCell, BioLegend, | | | | | | |
| beads***[SPB] | Biomagnetic X-GRAFFE [symbol] | | | | | | |
| Microfluidics | MicroMedicine Sorterra [symbol] | Poor | N/A | Slow | N/A | High | Low |
| | GPB Scientific Curate [symbol] | | | | | | |
| | Draper Labs [symbol] , Ferrologix [symbol] | | | | | | |
| Buoyancy/ | Akadeum Life Sciences [symbol] | Poor | N/A | Slow | Complex | High | Low |
| microbubbles | Diagnologix [symbol] | | | | | | |
| Specialized | Cobalt nano/graphene | | Medium | Medium | Complex | High | Low |
| material | TurboBeads [symbol] | | | | | | |
| | Aptamer-bound silica | Poor | N/A | Slow | Complex | High | Low |
| | Proteios Technology [symbol] | | | | | | |
| | Dissolvable hydrogel SPB | Poor | Low | Slow | Complex | High | Low |
| | Bio-Techne QuickGel [symbol] | | | | | | |

[symbol]

In development/research only [symbol]
indicates data missing or illegible when filed

As can be seen from Table 1, the beads that are used in connection with the present invention possess advantages in many areas relative to the beads of the prior art. Included in these properties and as shown in table 1 is the mixing ability of the beads, the magnetic strength of the beads, the speed with which various processes in connection with isolating granulocytes can occur, the elegant surface simplicity of the beads and the entire system, the good separation abilities (related to purity) and the high recovery abilities by using the beads and system of the present invention. Current immunomagnetic bead use recovers only <30%-70% of the desired cells for each depletion. In contrast, the use of the present magnetic beads retains greater than 97% of the therapeutic cells, while removing 99% of undesired cells. Thus, the magnetic beads of the present invention when the cell separation technology is used in cell therapy production, the quantity and concentration of desired starting cells will be enhanced. Furthermore, the ability of the magnetic beads of the present invention in purification of final therapeutic will lead to decreasing costs and broadening availability to cancer patients. Stated differently, the use of the magnetic beads as disclosed herein reduce manufacturing time, saving lives, therefore leading to cost reductions for both autologous and allogeneic cell therapies.

In an embodiment, the beads used herein also possess higher magnetic properties than the beads of the prior art (30-50 fold). A magnetic force is used to separate the bead/cell complex out of a biological mixture. Magnetic forces decrease by the square of the distance between the magnet and the particle of interest (volume processed). The more complicated (whole blood, vs apheresis sample, vs diluted, lysed sample) the more force (stronger magnet or set at a closer distance) and time required to separate a magnetically bound cell in a biological mixture.

The superparamagnetic beads of the prior art are limited by low magnetic susceptibility. They generally require specialized equipment or columns to place the magnetic force close (<6 mm) to the beads and they require reprocessing steps to simplify the complex mixture of cells and thereby reducing the drag on the bead. The columns and specialized equipment used for separation can place additional stress on cells, decreasing their functionality. The magnetic beads of the present invention, because they possess greater magnetism (up to 50 fold), do not suffer from these same drawbacks.

In contrast to the prior art where separation processes are performed multiple times when manufacturing cell therapeutics: first, to enrich the desired cell population from harvested material, and then second, during processing and final purification by removing non-therapeutic cells, viral and protein waste. Each time an immunomagnetic bead separation is performed using current technology, there is substantial loss of the desired or therapeutic cells. Losses can be as high as 70% each separation. Even if the loss is only 30%, when that process is used three times, the total yield drops down to 34%. The beads and processes used herein yield greater than 90% of the desired cells AFTER three separations (meaning that each separation has yields that vastly exceed 90%). Because of the improvements in yield seen by the present invention, the quantity of starting material required can be decreased (meaning patients do not have to give up as much blood). If the patient has a disease such as cancer, the reduction in blood quantity can be lifesaving.

The technology of the present invention allows for lower quantities to be used. However, the beads and processes of the present invention are well designed for larger quantities too. As alluded to above, because the magnetic beads have the ideal density (higher than the beads of the prior art) for undiluted blood, the beads can be properly dispersed throughout a larger quantity of blood (for example, in a blood bag) due to their densities. Because of their higher magnetic strength, the beads can be better isolated, separated without the use of special equipment. The combination of these two qualities allow the beads to be used and give very good and rapid dispersion, good and rapid binding to the undesired (or desired) component, and good and rapid separation ability.

One advantage to denser magnetic beads is that they are ideally suited to be dispersed throughout an undiluted blood bag. Thus, the beads can and will remain dispersed allowing for the possibility of longer binding times. This allows for slow kinetic (e.g., non-first order) interaction binding to proceed. Thus, the beads are ideally suited to situations in which the concentration of the undesired (or desired) component is low, or when the binding is not a very strong interaction. The magnetic strength of the beads subsequently allows them to be isolated rapidly allowing for much more precise times of binding.

The user also has significant control over the speed with which the magnetic beads can be pulled through the fluid medium. By applying a stronger external magnetic field, the beads can be moved more rapidly through the medium and by using a magnetic field of lesser strength, the magnetic beads can be move more slowly. It may be advantageous in certain conditions to move the magnetic beads more slowly through the fluid medium as the magnetic beads may still be able to bind the desired component while they are being pulled by the external magnetic field through the medium. This would afford the magnetic beads (while being pulled) a longer period of time to encounter and bind the desired component. The slower movement is also a more gentle approach and would prevent or reduce any possibility of shearing forces. Moving the beads more quickly by using a higher magnetic field strength reduces the amount of time that it takes, which in many instances may be a desired outcome. Magnetic field strengths that can be used are between 0.01 Tesla and 1.5 Tesla, with ranges that may be between 0.01 to 1 Tesla or 0.05 to 0.5 Tesla, or between 0.1 and 0.3 Tesla, or between 0.2 to 0.3 Tesla.

The plastic bags that are used for blood collection and/or collection of other fluids (e.g., urine) tend to be heat sealable. Accordingly, an embodiment of the present invention utilizes the heat sealability of the bags in order to effectively eliminate or greatly reduce granulocytes (or any other desired component) from undiluted blood (or any other fluid that is being collected). Denser beads can be added directly to the blood bags that contain an undiluted blood sample in it. The beads with the appropriately anti-granulocyte or anti-platelet molecule binds the granulocyte or platelet, respectively. The beads containing the unwanted cells bound to them can be pulled to a corner, side or bottom of the bag (using magnetic attraction, gravity, or a combination of the two). Once the beads with the undesired material is separated isolated from the rest of fluid in the bag, further processing can occur. For example, if the beads a present in a corner, the corner of the bag can be at least partially heat sealed so that the beads substantially remain in the corner of the bag. If the corner of the bag is not sealed completely but is left partly unsealed, the blood can be drained so as to release fluid that is not bound to the magnetic beads and this blood can amass with the other blood present in the bag, thereby allowing the corner to keep only the beads that have the undesired molecules/cells/products attached to them. This process will allow for very efficient removal of the unwanted product with very little of the fluid product lost. After drainage, the bag at that point can be sealed completely. In one variation, the beads (with the bound undesired material) may be excised completely from the bag by cutting the corner of the bag where they are present and sealing the bag completely (so that it is completely sealed).

In an embodiment, after isolating the magnetic beads that are bound to the granulocytes (or other component) in a corner, bottom, or side of the bag, the other blood (that is apart from the magnetic beads) can be optionally removed. In an embodiment, this blood can be removed via the use of a plunger or some other device (such as a syringe) and be moved to a different container (or for example, to another blood bag).

In an embodiment, the process may be automated so that a person merely has to place the bag in an apparatus that can perform the processes described herein automatically. In an embodiment, the magnetic beads can be recycled and used again in similar or different processes. It should be understood that this process describes removing unwanted components from fluid in bags (for example, prior to shipping) but this process can be used just as easily to isolate wanted components or to isolate components on which one can perform qualitative or quantitative tests.

Accordingly, in an embodiment, the present invention relates to a method of at least partially purifying an undiluted sample of blood in a heat sealing blood bag: the method comprising:
a) procuring an undiluted sample of blood and placing the undiluted sample of blood in the blood;
b) adding magnetic beads to the blood bag, wherein said magnetic beads have a first component affixed to the magnetic beads, said first component having an affinity for binding a second component;
c) allowing the first component to bind the second component so that the second component is operationally attached to the magnetic beads;
d) isolating the magnetic beads with the operationally attached second component in the heat sealing blood bag; and
e) optionally excising the magnetic beads with the operationally attached second component from the heat sealing blood bag.

In an embodiment, the method further comprises a step of at least partially sealing the heat sealing blood bag. The at least partially sealing the heat sealing blood bag may be performed to allow blood that is associated with the magnetic beads containing the first component and the second component attached to it to drain and amass with the other blood present in the blood bag.

In a variation, the second component is granulocytes or platelets. In a variation, the second component is granulocytes. In a variation, the second component is B-cells.

In a variation of the method, the method may further comprise an end step of completely sealing the heat sealing blood bag. This step may be a step that is performed prior to shipping the heat sealing blood bag, or prior to a secondary cell therapeutic processing step.

In an embodiment, the step of isolating the magnetic beads with the operationally attached second component in the heat sealing blood bag is performed by magnetism, gravity or a combination of magnetism is gravity.

In a variation, the excising step is performed by cutting the blood bag with a knife or scissors to excise the magnetic beads that are attached to the first and second component.

In a variation, the magnetic beads that are attached to the first and second component are recycled for use again.

In an embodiment, the present invention relates to a blood bag that comprises magnetic beads, said magnetic beads having a first component affixed to the magnetic beads, said first component having an affinity for binding a second component, wherein the magnetic beads are of a density that allows their even distribution throughout the blood bag when blood is present in the blood bag, the density of the magnetic beads being about 6-9 g/cc.

In a variation, the blood bag is made of a material that allows a magnet to move the magnetic beads to a corner of the blood bag so that the magnetic beads can be excised out of the bag by cutting the corner off of the bag. In a variation, the first component is an anti-granulocyte surface molecule (CD15) or an anti-granulocyte surface molecule (CD15) and one or more anti-platelet surface molecules (CD41 and/or CD61).

In a variation, the magnetic beads are lyophilized or present in an aqueous solution. In a variation, the blood bag is a heat sealing blood bag.

In an embodiment, the present invention relates to a kit comprising a blood bag and magnetic beads, said magnetic beads having a first component affixed to the magnetic beads, said first component having an affinity for binding a second component, wherein the magnetic beads are of a density that allows their even distribution throughout the blood bag when blood is present in the blood bag, the density of the magnetic beads being about 6-9 g/cc.

In a variation, the kit further comprises one or more of a magnet, instructions for separating granulocytes from blood, a pair of scissors, a heat source for heat sealing the blood bag, distilled water, distilled saline, a stirrer, a stir bar, a plunger, or a syringe.

In a variation, in the kit, the first component is an anti-granulocyte surface molecule (CD15) or an anti-granulocyte surface molecule (CD15) and one or more anti-platelet surface molecules (CD41 and/or CD61).

In an embodiment, the present invention relates to a method of preserving blood during transport, the method comprising using the blood bag and magnetic beads of the present invention during transport to remove granulocytes during transport. In a variation of this method, the method uses the features that are disclosed herein.

In an embodiment, the present invention relates to a system of purifying blood using the blood bag and the magnetic beads as disclosed herein.

In an embodiment, the present invention relates to a kit that comprises the magnetic beads of the present invention, a container such as a blood bag, instructions, a device that has or is capable of generating a magnetic field, and any of a plurality of other devices that can be used to practice the methods as disclosed/described herein. The device that has or is capable of generating/creating a magnetic field may have a magnet that is an aluminum nickel cobalt (AlNiCo) magnet, a samarium-cobalt magnet (SmCo), a neodymium magnet (Nd), a neodymium iron boron magnet (NdFeB), a ferromagnet, a ferrite magnet, a ceramic magnet, a cobalt magnet, a rare earth magnet, or an electromagnet.

The kit may comprise a blood bag and magnetic beads, said magnetic beads having a first component affixed to the magnetic beads, said first component having an affinity for binding a second component, wherein the magnetic beads are of a density that allows their even distribution throughout the blood bag when blood is present in the blood bag, the density of the magnetic beads being about 6-9 g/cc wherein the kit further comprises one or more of a magnet, instructions for separating granulocytes from blood, a pair of scissors, a heat source for heat sealing the blood bag, distilled water, distilled saline, a stirrer, a stir bar, a plunger, or a syringe, and wherein optionally, the first component is an anti-granulocyte surface molecule (CD15) or an anti-granulocyte surface molecule (CD15) and one or more anti-platelet surface molecules

(CD41 and/or CD61).

In an embodiment, one method of the present invention that can be practiced breaks the method down into four main groups of steps: these steps being 1) loading, 2) mixing, 3) separating, 4) and isolating. The loading step allows for the mixing of “dense” magnetic beads into a cellular mixture. In a variation, the loading step may be a preloaded wherein the magnetic beads are present in the container (e.g., a blood bag) prior to adding the cellular mixture. In an embodiment, there may be an injection port that allows the beads and/or the cellular mixture to be inserted into the container (e.g., blood bag).

In an embodiment, the mixing step allows the magnetic beads to move throughout the bag, including in a direction that is opposing gravity. This may be accomplished by using an external magnetic field and/or a mixer such as an end over end mixer, a magnetic stirrer, gas sparging, vortexing, or some other means. In an embodiment, the mixing step should be performed with variable mixing and not a uniform mixing motion/speed. For example, acceleration of mixing may adequately change during the process so as to provide more uniform dispersion throughout the container (e.g., blood bag). In an embodiment, the mixing should occur in a direction of any of the three axes. In an embodiment, the mixing step if done with gas sparging (e.g., using an inert gas), the head space should be limited to ⅓ of the overall total space if done in a flexible container, and at a minimum of ⅕ the total space for a rigid container.

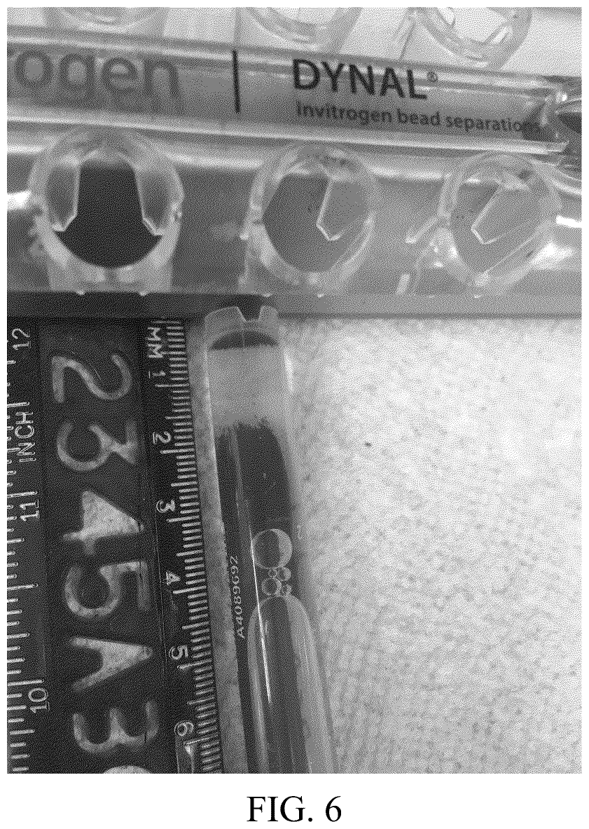

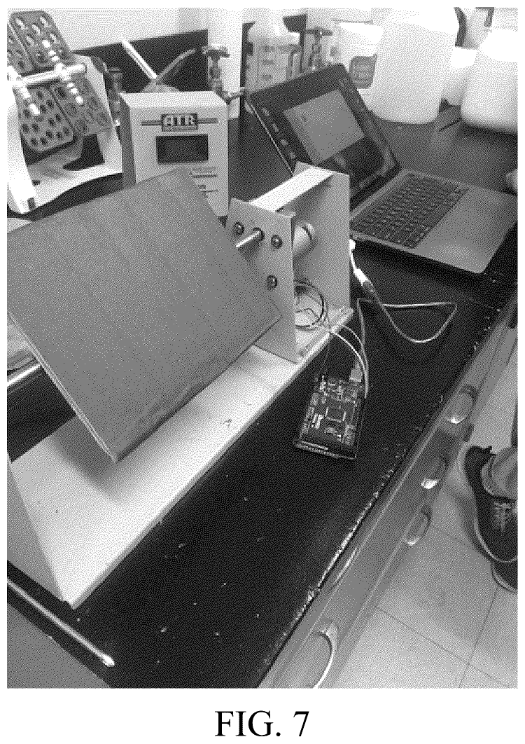

In an embodiment, the separating step provides a means of pulling by a magnetic field or by a metal that has magnetic properties, the magnetic beads distances that are as great as 4 or 5 cm. The separating step can be performed in any of a plurality of containers such as in a rigid container (e.g., bag) or a soft bag, which when performed allows the beads to be moved from a distance up to and including 4 cm. It should be noted that the beads can be “pulled” from a further distance when the magnetic field is stronger (i.e., a stronger magnet). The beads of the prior art often cannot be pulled through fluids of moderate viscosity because they have neither the density nor the compositional make-up sufficient to overcome the frictional or flowing forces of the fluid (e.g., they tend to remain suspended without the ability to be “pulled”). An example of the beads being pulled is shown in FIG. 6.

In an embodiment, the magnet that is used in the separating step is not very limited. For example, the magnet may be any of a plurality of shapes, including shaped in an array. It may be cylindrically or horizontally, or vertically oriented. It may be in the shape of a plate or like a magnetic stirrer, or alternatively, it may be bar shaped or a combination of shapes. In an embodiment, the magnet may be generated from a metal that has or is capable of generating/creating a magnetic field and the magnet may be an aluminum nickel cobalt (AlNiCo) magnet, a samarium-cobalt magnet (SmCo), a neodymium magnet (Nd), a neodymium iron boron magnet (NdFeB), a ferromagnet, a ferrite magnet, a ceramic magnet, a cobalt magnet, a rare earth magnet, an electromagnet, or combinations thereof.

In an embodiment, the isolating step is a means of removing certain cell components from a cellular mixture. The isolating step may use one or more immunomagnetic beads that allow the immunomagnetic beads to bind one or more components (e.g., cellular components) in the mixture (e.g., cellular mixture) and then to be isolated in any of a plurality of methods. For example, gravity may be used with the immunomagnetic beads and the residual fluid simply poured or siphoned off (or be removed by means of suction like using a pump, a syringe or plunger). An external magnetic field may alternatively and/or additionally be used to move the immunomagnetic beads to a particular location in the container (e.g., blood bag) and the beads excised and/or heat sealed to isolate the immunomagnetic beads (that have the desired component bound).

In an embodiment, the magnetic beads of the present invention may be supplied in a buffer solution and the beads and the buffer solution be mixed with the cellular mixture. In an alternate embodiment, when the magnetic beads of the present invention are “pulled” (i.e., moved by their magnetic properties) to remove them from the buffer solution, one may vortex or mix the beads prior to pulling the beads because they are “heavier” than the beads of the prior art, thereby requiring more vigorous agitation.

In an embodiment, the magnetic beads of the present invention may settle out from the cellular mixture (e.g., move to the bottom of the cellular mixture in a manner that is similar to a precipitate). In contrast, the prior art beads tend to remain suspended in the cellular mixture. The magnetic beads of the present invention thus afford more control to the user as the magnetic beads can be mixed into the cellular mixture as needed by one of the mixing techniques described herein (e.g., using gas or air sparging or end over end mixing). In contrast, the beads of the prior art tend to be suspended and cannot be readily moved throughout the cellular mixture (e.g., the prior art beads tend to remain in the same relative location no matter how vigorous the mixing is).

In an embodiment, because the magnetic beads of the present invention can be more readily moved as desired by the user, the methodology allows for more rapid separation, as well as better separation. The process tends to be rapid so that the magnetic beads of the present invention generally allow for good separation and/or isolation of components in small sample sizes (less than about 1-20 or 1-50 ml) in less than about 5 minutes. The lighter beads of the prior art simply cannot attain good separation in this amount of time. Their standard protocols require 45 mins. In an embodiment, if the sample size is small, a “head” or a “bubble” (i.e., a minimum of a 1 ml or 20% of the total volume is gas or air above the sample, whichever is less) may be important when the container is a tube shaped. The importance of the “head” or “bubble” is less important in a container that is a bag (such as a blood bag). However, in a bag, varying the speed of mixing appears to be important to get uniform dispersion of the magnetic beads in the cellular mixture. Without being bound by theory, it is believed that mixing at a constant speed creates a resonance for the beads that leaves the beads in the same relative location in the cellular mixture. In a variation, the magnetic beads should not just be spinning in a lateral direction when being mixed, but should also be moving vertically when mixed (otherwise the beads tend to settle). In an embodiment, the magnetic beads of the present invention work well in larger volumes (20-400 or 50-400 ml) because they are able to be pulled from a significantly further distance relative to the beads of the prior art. Generally, the beads of the prior art cannot be moved from a distance more than 0.6 mm. This severely limits the use of these beads to smaller volume sizes and also leads to poorer separation and/or isolation relative to the magnetic beads of the present invention. As disclosed elsewhere, the magnetic beads of the present invention, which are of a size between about 0.4 to 3.5 μm, because of their density and compositional make-up can be pulled when the beads are at a distance that is up to 4 or 5 cm or slightly more.

Moreover, the magnetic beads of the present invention are ideally suited to perform purification/separation/isolation of the targeted component(s) much more rapidly than the beads of the prior art. For example, the magnetic beads of the present invention can provide ideal purification/separation/isolation of the targeted component(s) in less than 45 minutes, or less than 40 minutes, or less than 35 minutes, or less than 30 minutes, or less than 20 minutes, or less than 10 minutes, or at about 5 minutes, or in a time less than about 5 minutes.

In an embodiment, because the beads of the present invention can be held with a magnet, this facilitates the ability to pour off (or drain) the residual fluid (e.g., cellular mixture) to another container, or potentially another section of the same container. In a variation, this separation/isolation procedure can be performed rapidly and without the use of centrifugal forces. Alternatively, and/or additionally, the beads can be separated by use of a heat-sealing bag.

The following references are herein incorporated by reference for all purposes.

### CITED REFERENCES

### Patents

U.S. Pat. No. 5,262,302 Russell Nov. 16, 1993
U.S. Pat. No. 5,576,185 Coulter Nov. 19, 1996
U.S. Pat. No. 5,653,686 Coulter Aug. 5, 1997
U.S. Pat. No. 9,435,799 Russell Sep. 6, 2016
U.S. Pat. No. 9,739,768 Russell Aug. 23, 2017
U.S. Pat. No. 5,466,574 Liberti Nov. 4, 1995
U.S. Pat. No. 5,411,863 Miltenyi May 2, 1995
U.S. Pat. No. 4,654,267 Ugelstad Mar. 31, 1987
U.S. Pat. No. 4,707,523 Chang Nov. 17, 1987

### Other Publications

1. Bull M, Lee D, Stucky J, Chiu Y L, Rubin A, Horton H, et al. Defining blood processing parameters for optimal detection of cryopreserved antigen-specific responses for HIV vaccine trials. J Immunol Methods. 2007 Apr. 30; 322(1-2):57-69.
2. DeRose, R et al. Granulocyte contamination dramatically inhibits spot formation in AIDS virus-specific ELISpot assay. J. Immuno Methods. 2005 297; 177-186.
3. Kierstead L S, Dubey S, Meyer B, Tobery T W, Mogg R, Fernandez V R, et al. Enhanced rates and magnitude of immune responses detected against an HIV vaccine: effect of using an optimized process for isolating PBMC. AIDS Res Hum Retroviruses. 2007 January; 23(1):86-92.
4. Kim D W, Jang, Y Y et al. Overnight Storage of Blood in ACD Tubes at 4 deg C. increases NK Cell Function in PBMC. Annals of Clinical and Lab Sciences. 2013; 43; 267.
5. Mallone, R, Mannering, S I et al. Isolation and preservation of peripheral blood mononuclear cells for analysis of islet antigen-reactive T cell responses: position statement of the T-Cell Workshop Committee of the Immunology of Diabetes Society. Clinical and Experimental Immunology. 2010; 163. 33-49
6. McKenna K C, Beatty K M, Vicetti Miguel R, Bilonick R A. Delayed processing of blood increases the frequency of activated CD11b+ CD15+ granulocytes which inhibit T cell function. Journal of Immunological Methods. [Comparative Study]. 2009 Feb. 28; 341(1-2):68-75.
7. Preobrazhensky, S N and Bahler, D W. Immunomagnetic bead separation of mononuclear cells from contaminating granulocytes in cryopreserved blood sample. Cryobiology 2009; 59; 366-368
8. Miltenyi Catalog: Order no: 130-093-545
9. ThermoFisher Catalog: Catalog number: 11137D DYNAL CD15 magnetic beads
10. Zhao, Z.; Fan, J.; Hsu, Y. S.; Lyon, C. J.; Ning, B.; Hu, T. Y. Extracellular vesicles as cancer liquid biopsies: From discovery, validation, to clinical application. Lab Chip 2019, 19, 1114-1140.

We claim:

1. A method of at least partially purifying or removing a second component from a fluid, the method comprising:
a) procuring an undiluted fluid that contains a second component and placing the undiluted fluid in a container,
b) adding magnetic beads to the container, wherein said magnetic beads have a first component affixed to the magnetic beads, said first component having an affinity for binding the second component,
c) allowing the first component to bind the second component so that the second component is operationally attached to the magnetic beads,
d) isolating the magnetic beads with the operationally attached second component in the container from a residual fluid that has some of the second component removed,
e) optionally separating the magnetic beads with the operationally attached second component from the residual fluid, and
f) optionally performing said method in less than about 40 minutes.

2. The method of claim 1, wherein the isolating occurs by applying an external magnetic field to the container.

3. The method of claim 1, wherein the method further comprises a mixing step wherein the fluid in the container is mixed to allow binding of the first component to the second component.

4. The method of claim 3, wherein the mixing step uses a mixer that mixes in a direction of that is opposite gravity.

5. The method of claim 4, wherein the mixing step utilizes variable mixing.

6. The method of claim 5, wherein the variable mixing causes an even distribution of the magnetic beads throughout a whole of the fluid in the container.

7. The method of claim 1, wherein a viscosity of the fluid is at least about 3 cP.

8. The method of claim 7, wherein the viscosity of the fluid is 6 cP or less.

9. The method of claim 1, wherein the magnetic beads are a density that is between about 6-9 g/cc.

10. The method of claim 1, wherein the separating the magnetic beads is done by gravity and/or by an external magnetic field.

11. The method of claim 1, wherein the fluid is an undiluted sample of blood and the container is a heat sealing blood bag: wherein the method comprises:
a) procuring an undiluted sample of blood and placing the undiluted sample of blood in the heat sealing blood bag,
b) adding magnetic beads to the heat sealing blood bag, wherein said magnetic beads have a first component affixed to the magnetic beads, said first component having an affinity for binding a second component,
c) allowing the first component to bind the second component so that the second component is operationally attached to the magnetic beads,
d) isolating the magnetic beads with the operationally attached second component in the heat sealing blood bag,
e) optionally excising the magnetic beads with the operationally attached second component from the heat sealing blood bag.

12. The method of claim 11, further comprising a step of at least partially sealing the heat sealing blood bag.

13. The method of claim 11, wherein the second component is granulocytes or platelets.

14. The method of claim 11, further comprising an end step of completely sealing the heat sealing blood bag.

15. The method of claim 11, wherein the isolating the magnetic beads with the operationally attached second component in the heat sealing blood bag is performed by magnetism, gravity or a combination of magnetism and gravity.

16. The method of claim 15, wherein the isolating the magnetic beads with the operationally attached second component in the heat sealing blood bag is performed by magnetism.

17. The method of claim 11, further comprising a mixing step wherein the mixing is by one or more of the following: sparging with a gas, vortexing, shaking, using a magnetic stir bar, or inversion of the heat sealing blood bag.

18. The method of claim 11, wherein the mixing is accomplished by mixing at variable speeds.

19. A blood bag that comprises magnetic beads, said magnetic beads having a first component affixed to the magnetic beads, said first component having an affinity for binding a second component, wherein the magnetic beads are of a density that allows their even distribution throughout the blood bag when blood is present in the blood bag, the density of the magnetic beads being about 6-9 g/cc.

20. A kit comprising a blood bag and magnetic beads, said magnetic beads having a first component affixed to the magnetic beads, said first component having an affinity for binding a second component, wherein the magnetic beads are of a density that allows their even distribution throughout the blood bag when blood is present in the blood bag, the density of the magnetic beads being about 6-9 g/cc wherein the kit further comprises one or more of a magnet, instructions for separating granulocytes from blood, a pair of scissors, a heat source for heat sealing the blood bag, distilled water, distilled saline, a stirrer, a stir bar, a plunger, or a syringe, and wherein optionally, the first component is an anti-granulocyte surface molecule (CD15) or an anti-granulocyte surface molecule (CD15) and one or more anti-platelet surface molecules (CD41 and/or CD61).
